(19) United States
(12) Patent Application Publication
(10) Pub. No.: US 2024/0173533 A1
(43) Pub. Date: May 30, 2024

(54) Open Female Luer Disinfecting Cap

(71) Applicant: Becton, Dickinson and Company, Franklin Lakes, NJ (US)

(72) Inventors: Manish Kumar, Karnataka (IN); Karthik MR, Bangalore (IN); Praveen Nalawade, Belagavi (IN); Shishir Prasad, Ramsey, NJ (US)

(21) Appl. No.: 18/070,828

(22) Filed: Nov. 29, 2022

(51) Int. Cl. A61M 39/16; A61M 39/20

(52) U.S. Cl. CPC A61M 39/162; A61M 39/20

(57) ABSTRACT

A disinfecting cap configured to engage an open female luer connector includes a housing having an open first end, a second end, and at least one sidewall extending between the first end and the second end. The cap also includes an absorbent support or sponge disposed in the housing configured to contain a cleaning solution for cleaning and/or disinfecting portions of the open female luer connector engaged to the cap. The cap further includes a plug disposed in the housing configured to prevent ingress of the cleaning solution into a lumen in the open female luer connector.

### BACKGROUND OF THE INVENTION

### Field of the Invention

The present disclosure relates generally to caps for medical connectors and, in particular, to a medical cap configured to be attached to open female luer connectors for sealing, cleaning, and disinfecting portions of the open female luer connectors.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Further, bacteria and other microorganisms may enter into a patient's vascular system from access hubs, ports, or valves upon connection to the VAD to deliver a fluid or pharmaceutical to a patient. Therefore, each access hub, port, valve, or other connection configured for attachment to a VAD is associated with some risk of transmitting a catheter related bloodstream infection (CRBSI) to a patient.

Many medical facilities implement sterile practices and protocols to ensure that VADs and access hubs or ports are used properly and do not become sealed or infected. These protocols often include sterilizing the access hubs, ports, and VADs, as well as flushing the catheter with a flush solution prior to use. Specifically, VAD standards of practice usually recommend flush procedures be performed after catheter placement, before fluid infusion, and before and after drug administration, blood sampling, transfusions, and/or administration of parenteral nutrition.

Standards of practice can also require that access hubs, ports, and valves be capped with disinfection caps when not in use, to prevent microbial ingress into the hub, port, or valve and to sterilize areas of the hub, port, or valve that contact the VAD. Disinfection caps are disposable cap devices that contain an amount of cleaning or disinfecting solution for sterilizing portions of the port, hub, and valve. Accordingly, disinfecting caps can be both a barrier preventing microbes, particles, and debris from contacting portions of hubs, ports, or VADs that should remain sterile and disinfecting or cleaning devices configured to expose portions of the hubs, ports, and VADs to cleaning and disinfecting solutions.

Open female luers do not have any septum and hence need to be blocked or plugged to reduce cleaning or disinfecting solution, typically Isopropyl alcohol (IPA), ingress into the fluid path. However, conventional disinfecting caps for open female luer connectors may not prevent ingress of the disinfectant into the lumen of the open female luer connectors and in turn the fluid path within the open female luer. The disinfecting caps of the present disclosure are configured to address this issue and effectively block ingress of IPA into the lumen of a female luer connection.

### SUMMARY OF THE INVENTION

According to an aspect of the disclosure, a cap configured to engage an open female luer connector includes a housing having an open first end, a second end, and at least one sidewall extending between the first end and the second end defining a recess. The cap also includes an absorbent support or sponge which retains disinfectant such as Isopropyl alcohol (IPA) in an open cell structure that can be released when the plug is compressed by the open female luer connection for cleaning and/or disinfecting portions of the open female connector engaged to the cap. The cap may also include a bellow and/or a plug disposed in the recess of the housing that enclose the sponge but include openings to allow for disinfectant to move out from the sponge and onto outer surfaces of the open female luer connection. The plug may be configured to block ingress of the IPA into the lumen of the open female luer connection.

According to another aspect of the disclosure, the plug may be configured to allow the lumen of the open female luer to be blocked prior to connection of the open female luer with the cap. The cap may be rigid. The sponge attached to bellow and/or plug may be configured to allow IPA to be released as the connection is made between the cap threads and the open female luer progresses. The luer distal face and outer surfaces may thus be wetted by released IPA and hence disinfection of the open female luer is attained.

According to another aspect of the disclosure, a method for attaching the previously described cap to the open female luer connector includes a step of inserting a distal open end of the open female luer connector through the open first end of the housing causing the distal open end of the open female luer connector to contact the plug disposed in the housing over the absorbent support thereby blocking or sealing the lumen in the open female luer connector. The method also includes a step of rotating the open female luer connector relative to the housing causing the open distal end of the open female luer connector to advance through the housing axially compressing the absorbent support thereby releasing cleaning solution into the housing such that portions of the open female luer connector outside of the lumen may be exposed to the cleaning solution.

According to another aspect of the disclosure, this device can comprise a rigid cap, sponge, bellow, plug and IPA. The rigid cap may be a plastic cap which houses the various components within it. The sponge may retain IPA in an open cell structure and release it when compressed by an incoming open female luer connector. The bellow may enclose the sponge and may further comprise cutouts/slits in its lateral body which allows IPA to move out from the sponge to the space between the bellow and inner surface of rigid cap. The material of construction can be but is not limited to thermoplastic, elastomer, silicone, and/or rubber among other materials. The plug may act as a sealing element to the inner lumen of the open female luer connection. This can restrict IPA ingress into the lumen and in turn into the fluid path of the open female luer connection. IPA may be filled in the cap, and most of which is retained in the sponge, and acts as a disinfectant. The bellow and the sponge can be bonded to the bottom inner base of the rigid cap.

According to another aspect of the disclosure, when an open female luer-like catheter hub or stopcock needs to be disinfected and protected, the top web peel film of the cap is removed, the cap is attached to the female luer, the bellow and/or plug first enters the inner lumen of the female luer connector and seals the lumen thereby not allowing IPA from the cap to ingress into the fluid path in the female luer connector. As connection between the cap and the female luer connector progresses, the plug and/or bellow is compressed, and the sponge attached to plug and/or bellow may also compress and release IPA from an open cell structure. The released IPA may wet the distal front face of the open female luer connection as well as the luer sides and disinfects it due to chemical action of IPA. The cap may finally be connected onto the female luer connector using a threaded connection and can remain connected for up to 7 days if not removed.

In accordance with an embodiment of the present invention, a disinfectant cap configured to engage an open female luer connector including a lumen, includes a housing having an open first end, a second end, and at least one sidewall extending between the first end and the second end defining a recess; a plug disposed in the recess of the housing configured to engage the open female luer connector for sealing the lumen of the open female connector; and an absorbent support disposed in the recess of the housing configured to contain a cleaning solution for cleaning and/or disinfecting portions of the open female luer connector engaged to the cap.

In accordance with an embodiment of the present invention, the absorbent support includes a sponge.

In accordance with an embodiment of the present invention, the cleaning solution comprises as an example but not limited to or any combination of Isopropyl Alcohol (IPA), chlorhexidine, and povidone-iodine.

In accordance with an embodiment of the present invention, when the open female luer connector is engaged to the cap, cleaning solution is exposed to portions of the open female luer connector for cleaning and/or disinfecting the open female luer connector.

In accordance with an embodiment of the present invention, the open female luer connector further includes an outer surface and a thread extending from the outer surface and the sidewall of the housing further comprises an inner surface that comprises threads to engage the thread on the outer surface open female luer connector.

In accordance with an embodiment of the present invention, the cap further includes a bellow having a first open end, a second open end, and a sidewall defining a cylindrical opening that is configured to enclose the absorbent support wherein the first open end of the bellow is configured to connect to the plug to further enclose the absorbent support.

In accordance with an embodiment of the present invention, the bellow further includes a sidewall having ridges and troughs configured to be compressed and a plurality of openings in the sidewall configured to allow cleaning solution to flow from the absorbent support into the recess formed by the housing to clean and disinfect portions of the open female luer connector when the absorbent support is compressed by the insertion of the open female luer connector into the cap.

In accordance with an embodiment of the present invention, the cleaning solution includes but is not limited to one of or any combination of Isopropyl Alcohol (IPA), chlorhexidine, and povidone-iodine and the absorbent support comprises a sponge.

In accordance with an embodiment of the present invention, the plug includes a first disc configured to fit within and seal the lumen of the open female luer connector.

In accordance with an embodiment of the present invention, the plug further includes a second disc having a larger diameter than the first disc and connected to the first disc wherein the second disc comprises a plurality of holes for permitting the cleaning solution to move past the plug towards the open first end of the housing.

In accordance with an embodiment of the present invention, the plug includes a hemispherical member configured to fit within and seal the lumen of the open female luer connector.

In accordance with an embodiment of the present invention, the plug further includes a cylindrical member with a first end affixed to the hemispherical member and a second end slidably engaged to a plug guide comprising a top open end for receiving the second end of the cylindrical member of the plug and a bottom second end connected to the second end of the housing.

In accordance with an embodiment of the present invention, the absorbent support includes an open top end, an open bottom end, and sidewall extending between the top and bottom ends defining an inner cylindrical opening configured to receive and surround portions of the plug.

In accordance with an embodiment of the present invention, the plug includes a cylindrical member having a first end configured to fit within and seal the lumen of the open female luer connector and a second end having a larger diameter than the first end and the lumen configured to further engage the open female luer connector.

In accordance with an embodiment of the present invention, the absorbent support includes an open top end, an open bottom end, and sidewall extending between the top and bottom ends defining an inner cylindrical opening configured to receive and surround portions of the second end of the plug.

In accordance with an embodiment of the present invention, insertion of the open female luer connector into the housing causes the absorbent support to axially compress farther into the housing, and wherein the axial compression of absorbent support expels the cleaning solution from the absorbent support causing the cleaning solution to move through an interior of the housing contacting an outer surface of the open female luer connector inserted into the housing.

In accordance with an embodiment of the present invention, a method for attaching a cap to the open female luer connector includes inserting a distal open end of the open female connector through the open first end of the housing causing the proximal end of the open female connector to contact the plug disposed in the housing and the absorbent support; and rotating the open female luer connector relative to the housing causing the proximal end of the open female luer connector to advance through the housing axially compressing the absorbent support.

In accordance with an embodiment of the present invention, a cap configured to engage an open female connector having a lumen includes a housing having an open first end, a second end, and at least one sidewall extending between the first end and the second defining a recess; a bellow comprising an open first end, an open second end, and at least one sidewall extending between the first end and the second end defining a cavity disposed in the recess defined by the housing; an absorbent support disposed in the cavity defined by the bellow and contained by the sidewall of the bellow configured to contain a cleaning solution for cleaning and disinfecting portions of the female connector engaged to the cap; a plug connected to the open first end of the bellow configured to further contain the absorbent support and prevent the cleaning solution from entering the lumen of the female connector.

In accordance with an embodiment of the present invention, the absorbent support includes a sponge and the cleaning solution comprises for example one of or any combination of Isopropyl Alcohol (IPA), chlorhexidine, and povidone-iodine.

In accordance with an embodiment of the present invention, a cap configured to engage a female connector having a lumen includes a rigid housing comprising an open first end, a second end, and a sidewall extending between the first end and the second defining a recess; a sponge disposed in the recess defined by the housing configured to contain disinfecting liquid such as Isopropyl Alcohol (IPA) for cleaning and disinfecting portions of the female connector engaged to the cap; a plug configured to be inserted into the lumen of the female connector to prevent IPA from entering the lumen of the female connector.

In accordance with an embodiment of the present invention, a cap is configured to engage an open female connector having a lumen, the cap including a housing having an open first end, a second end, and at least one sidewall extending between the first end and the second defining a recess; a bellow comprising an open first end, an open second end, and at least one sidewall extending between the first end and the second end defining a cavity disposed in the recess defined by the housing; an absorbent support disposed in the cavity defined by the bellow and contained by the sidewall of the bellow configured to contain a cleaning solution for cleaning and/or disinfecting portions of the female connector engaged to the cap; a plug connected to the open first end of the bellow configured to further contain the absorbent support and prevent the cleaning solution from entering the lumen of the female connector.

In accordance with an embodiment of the present invention, the open female connector includes an open female luer connector.

In accordance with an embodiment of the present invention, the absorbent support includes a sponge.

In accordance with an embodiment of the present invention, the plug includes at least one circular disc configured to block ingress of cleaning solution into the lumen of open female luer connector.

In accordance with an embodiment of the present invention, when the open female luer connector is engaged to the cap, a portion of the open female luer connector is inserted into the recess defined by the housing and a portion of the plug is in inserted into the lumen of the female connector.

In accordance with an embodiment of the present invention, when the open female luer connector is engaged to the cap, cleaning solution is exposed to portions of the open female luer connector for cleaning and/or disinfecting those portions of the open female luer connector.

In accordance with an embodiment of the present invention, the cap is sized to receive open female luer connectors having different configurations and dimensions.

In accordance with an embodiment of the present invention, the housing includes a rigid thermoplastic polymer comprising at least one of polyester, polycarbonate, polypropylene, polyethylene, polyethylene terephthalate, or acrylonitrile butadiene styrene.

In accordance with an embodiment of the present invention, the housing includes a plurality of ridges or ribs extending outwardly from an outer surface of the housing (for making the housing easier to grip).

In accordance with an embodiment of the present invention, the absorbent support or sponge is axially compressible.

In accordance with an embodiment of the present invention, the recess defined by the housing has an inner diameter corresponding to an outer diameter of the absorbent support or sponge.

In accordance with an embodiment of the present invention, the absorbent support or sponge includes a cylindrical member that is axially compressible.

In accordance with an embodiment of the present invention, the absorbent support or sponge includes a thermoplastic elastomer such as silicone, polypropylene, polyethylene, or synthetic or natural rubber (e.g., isoprene).

In accordance with an embodiment of the present invention, the bellow includes an open first end, an open second end, and a sidewall extending between the first end and the second end defining a cavity dimensioned to enclose the absorbent support or sponge.

In accordance with an embodiment of the present invention, the sidewall of the bellow is formed to include a plurality of ridges extending radially outward from the sidewall of the bellow, the ridges configured to deform outward when the bellow is compressed.

In accordance with an embodiment of the present invention, the housing further includes a plurality of threads disposed on the inner surface of the sidewall of the housing configured to engage a portion of the open female luer connector and rotatably secure the open female luer connector within the housing.

In accordance with an embodiment of the present invention, the sidewall of the bellow includes at least one opening for permitting the cleaning solution from the absorbent support to pass through the bellow into the recess defined by the opening.

In accordance with an embodiment of the present invention, the plug is formed from a same rigid thermoplastic polymer as the housing.

In accordance with an embodiment of the present invention, the absorbent support includes a thermoplastic elastomer such as silicone, polypropylene, polyethylene, or synthetic or natural rubber (e.g., isoprene).

In accordance with an embodiment of the present invention, the absorbent support includes a sponge.

In accordance with an embodiment of the present invention, the absorbent support includes an open cell foam, such as a porous foam having a thermoplastic elastomer.

In accordance with an embodiment of the present invention, insertion of the open female luer connector into the housing causes the absorbent support to axially compress farther into the housing.

In accordance with an embodiment of the present invention, axial compression of the absorbent support expels the cleaning solution from the inner absorbent support causing the cleaning solution to move through an interior of the housing contacting an outer surface of the open female luer connector inserted in the housing.

In accordance with an embodiment of the present invention, the cleaning solution may comprise Isopropyl Alcohol (IPA), chlorhexidine, or povidone-iodine.

In accordance with an embodiment of the present invention, the plug is disposed in the housing over the absorbent support and is configured to seal an open end of the open female luer connector, thereby preventing the cleaning solution from entering the lumen of the open female connector.

In accordance with an embodiment of the present invention, the plug is positioned to permit the cleaning solution expelled from the absorbent member to move past the plug towards the first open end of the housing when the absorbent member is axially compressed.

In accordance with an embodiment of the present invention, an outer diameter of the plug is less than an inner diameter of the housing.

In accordance with an embodiment of the present invention, the plug includes at least one of holes, cut-away portions, grooves, slits, or slots for permitting the cleaning solution to move past the plug towards the open first end of the housing while the plug blocks the cleaning solution from entering the lumen of the open female luer connector.

In accordance with an embodiment of the present invention, a method for attaching a cap to the open female luer connector includes inserting a distal open end of the open female luer connector through the open first end of the housing causing the distal open end of the open female luer connector to contact a plug disposed in the housing adjacent an absorbent support; and rotating the open female luer connector relative to the housing causing the distal open end of the open female luer connector to advance through the housing axially compressing the absorbent support.

In accordance with an embodiment of the present invention, advancing the distal open end of the open female luer connector through the housing causes the cleaning solution to be expelled from the absorbent support and to move through the housing contacting an outer surface of the open female luer connector.

In accordance with an embodiment of the present invention, contact between the distal open end of the open female luer connector and the plug seals the distal open end of the open female luer connector preventing the cleaning solution from passing into a lumen of open female luer connector.

In accordance with an embodiment of the present invention, upon axial compression of the absorbent support, the cleaning solution moves past the plug towards the open first end of the housing contacting an outer surface of the distal open end of the open female luer connector.

In accordance with an embodiment of the present invention, a manufacturing method for a cap includes inserting the absorbent support into the recess of the housing; and inserting the plug adjacent the absorbent support.

In accordance with an embodiment of the present invention, the housing includes a rigid thermoplastic polymer having at least one of polyester, polycarbonate, polypropylene, polyethylene, polyethylene terephthalate, or acrylonitrile butadiene styrene.

### BRIEF DESCRIPTION OF THE DRAWINGS

FIG. 1 is a cross-sectional view of an open female luer connector as is known in the prior art.

FIG. 2 is a cross-sectional view of a disinfecting cap connected to an open female luer connector, according to an aspect of the present disclosure.

FIG. 3 is an exploded perspective view of the cap of FIG. 2, according to an aspect of the present disclosure 1.

FIG. 4 is an exploded cross-sectional view of the cap of FIG. 2, according to an aspect of the present disclosure.

FIG. 5 is perspective view of the bellows and cap of FIG. 2, according to an aspect of the present disclosure.

FIG. 6A is an exploded cross-sectional view of the cap of FIG. 2 and an open female luer connector, prior to inserting the male connector into the cap.

FIG. 6B is a perspective view of the cap of FIG. 2 with the open female luer connector inserted into the cap.

FIG. 6C is a cross-sectional view of the disinfecting cap of FIG. 2 with the open female luer connector inserted into the cap.

FIG. 7 is a cross-sectional view of another example of a disinfecting cap connected to an open female luer connector, according to another aspect of the present disclosure.

FIG. 8 is an exploded perspective view of the disinfecting cap of FIG. 7.

FIG. 9 is an exploded cross-sectional view of the disinfecting cap of FIG. 7.

FIG. 10 is a perspective view of a plug that may comprise a disinfecting cap as shown in FIGS. 2-9, according to another aspect of the present disclosure.

FIG. 11A is a cross-sectional view of the disinfecting cap of FIG. 7 and an open female luer connector just prior to insertion of the open female luer connector into the cap.

FIG. 11B is a cross-sectional view of the disinfecting cap of FIG. 7 and an open female luer connector after partial insertion of the open female luer connector into the cap.

FIG. 12 is a cross sectional view of another example of a disinfecting cap connected to an open female luer connector, according to another aspect of the present disclosure.

FIG. 13 is an exploded perspective view of the cap of FIG. 12.

FIG. 14 is an exploded cross-sectional view of the cap of FIG. 12.

FIG. 15 is a cross-sectional view of the cap of FIG. 12 with the open female luer connector partially inserted into the cap.

FIG. 16 is a cross-sectional view of the cap of FIG. 12 with the open female luer connector more fully inserted into the cap.

FIG. 17 is a cross sectional view of another example of a disinfecting cap connected to an open female luer connector, according to another aspect of the present disclosure.

FIG. 18 is an exploded perspective view of the cap of FIG. 17.

FIG. 19 is an exploded cross-sectional view of the disinfecting cap of FIG. 17.

FIG. 20A is a cross-sectional view of the cap of FIG. 17 with the open female luer connector just prior to the insertion of open female luer connector into the cap.

FIG. 20B is a cross-sectional view of the cap of FIG. 17 with the open female luer connector partially inserted into the cap.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. The term “proximal” refers to a portion of a device or part that is grasped by a user or connected to another device or part. The term “distal” refers to a portion of a device or part that is opposite the proximal portion (i.e., farthest away from the portion that is grasped by a user or connected to another part). For example, for an implantable medical device, such as a catheter, a proximal portion can refer to the portion of the catheter that remains outside of a patient's body and is manipulated by a user. The distal portion of the catheter can be the portion that is inserted into the vasculature of the patient. For an object or part, such as an elongated member, the proximal end can be the end that is connected to another object or part. The distal end of the elongated member is opposite the proximal end and can be free from connections to other objects or parts. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a disinfecting cap 10 configured to be connected to an open female luer connector 112, such as an open female luer connector of an access hub, port, or valve for a VAD, to prevent the connector, port, or VAD from being contaminated by, for example, microbes, debris, or other contaminants. In some examples, the cap 10 can be configured to clean or disinfect portions of the connector 112, ensuring that the connector 112 remains sterile prior to use. The cap 10 can be configured to remain in place on a connector 112 for at least seven days, which is a maximum time of recommended use permitted by many medical facility sterile practice guidelines.

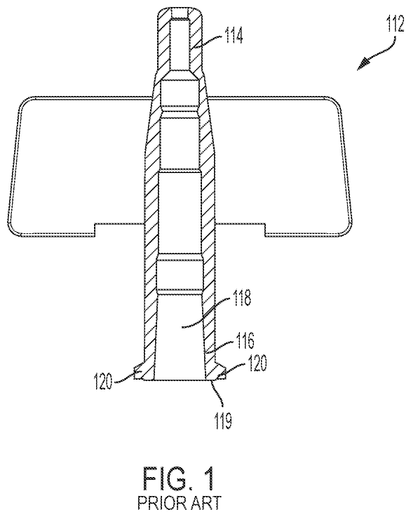

The cap 10 is a disinfecting cap for open female luer connectors 112 configured to engage with or be connected to different sizes, configurations, and/or types of connectors 112. The open female luer connector 112 can comprise an elongated distal end portion 114 and an open distal end 116 that is of a larger diameter than the distal end portion 114 with a tapered lumen 118 extending from the distal end portion 114 to the open distal end 116 through the center of the connector 112. An engaging structure, such as thread or luer lug 120, may extend from the outside surface of the open distal end 116. An exemplary open female luer connector 112 in accord with the prior art is shown in FIG. 1.

### Disinfecting Cap for Open Female Luer Connectors

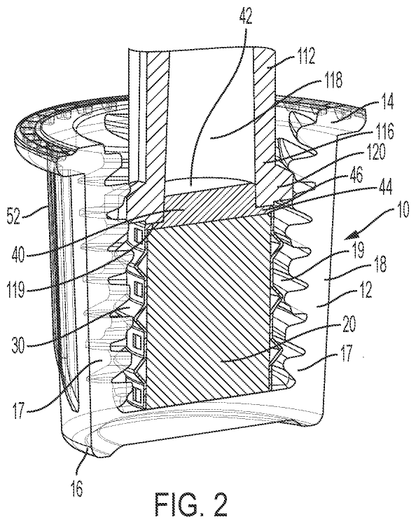

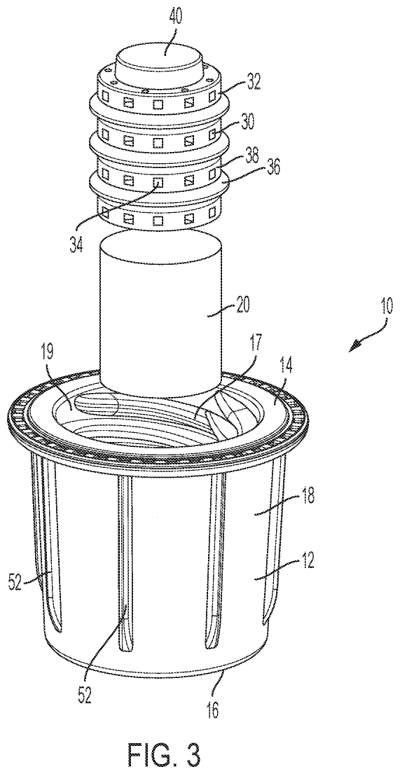

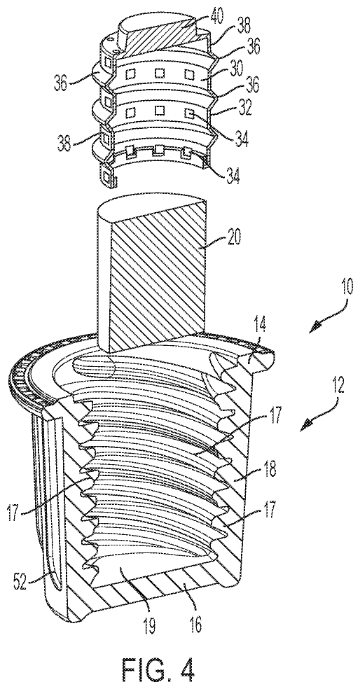

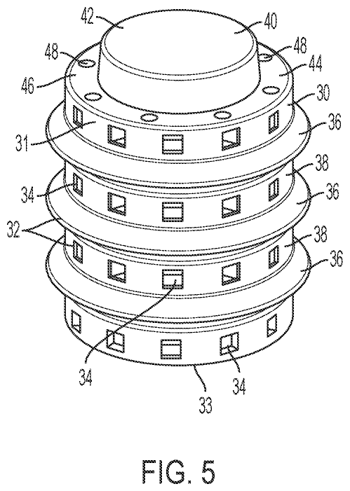

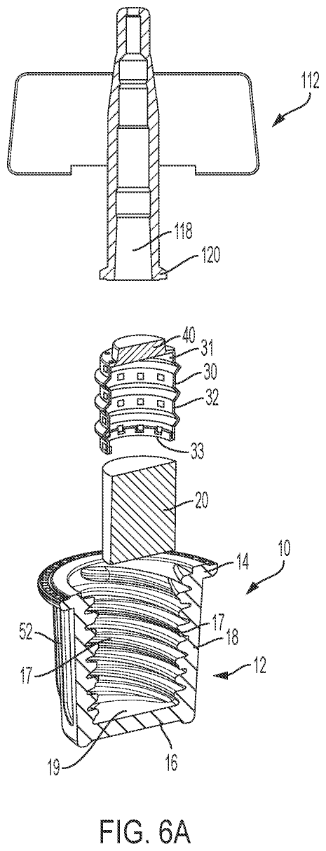

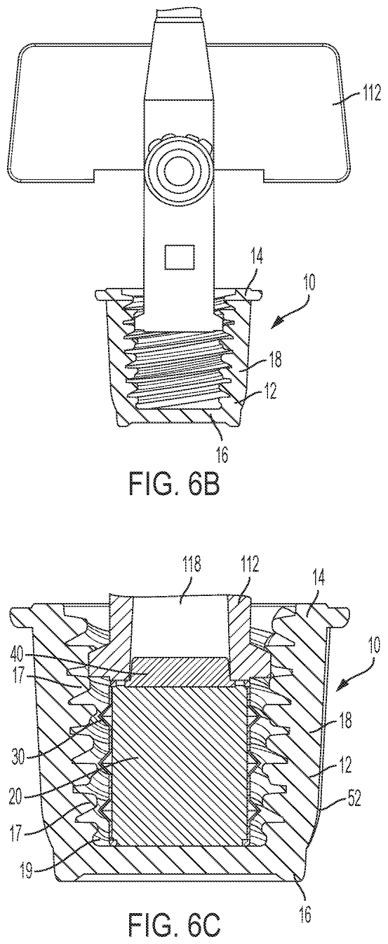

FIGS. 2A-6C illustrate portions of an exemplary disinfecting cap 10 configured to engage and/or to be connected to open female luer connectors 112 with different shapes, sizes, dimensions, or configurations. Specifically, FIG. 2 is a cross-sectional view of the cap 10 connected to an open female luer connector 112. FIG. 3 is an exploded view of the cap 10. FIG. 4 is an exploded cross-sectional view of the cap 10. FIG. 5 is a perspective view of a bellow and a plug of the cap 10. FIG. 6A is an exploded cross-sectional view of the cap 10 and the open female luer connector 112 before insertion. FIG. 6B is a perspective view showing the open female luer connector 112 inserted into the disinfecting cap 10. FIG. 6C is a cross-sectional view showing the open female luer connector 110 inserted into the disinfecting cap 10.

In some examples, the cap 10 can be provided as a single pre-packaged cap or cap assembly as shown in exploded view in FIG. 3. Further, as described in detail herein, the cap 10 includes components, such as an absorbent support or sponge and/or cleaning or disinfecting solutions, for cleaning and disinfecting portions of the open female luer connectors 112 inserted into and mounted to the cap 10.

As shown in FIGS. 2A-6C, the cap 10 comprises a rigid cap or housing 12 comprising an open first end or top 14, a second end or bottom 16, and a sidewall 18 extending between the top 14 and the bottom 16 forming a cavity 19. The rigid cap 12 may be constructed of plastic. The cap 10 further comprises an absorbent support 20, which can comprise sponge 20 in some examples, enclosed by a bellow 30 and a plug 40 and disposed within the recess or cavity 18 of the rigid cap 12. The absorbent support or sponge 20 can be a cylindrical member sized to fit within the bellow 30 and cap 40 which are sized to fit within the recess or cavity 19 of the rigid cap 12. The rigid cap or housing 12 also comprises threads 17 disposed on the inner surface of the sidewall 18 configured to engage the thread or luer lug 120 on the open female luer connector 112 for retaining the connector 112 within the rigid cap or housing 12.

The absorbent support or sponge 20 comprises disinfectant, such as IPA, for cleaning distal portions 116 of the open female luer male connector 112 when the connector 112 is engaged to the cap 10. The absorbent support or sponge 20 may be comprised of an open cell structure to retain IPA and release IPA when the absorbent support or sponge 20 is compressed by an incoming open female luer connector 112 when it is engaged to the cap 10, or more specifically, when the thread or luer lug 120 on the open female luer connector 112 engages the threads 17 on the rigid cap or housing 12.

The absorbent support or sponge 20 may be disposed within a bellow 30. The bellow 30 may comprise a tubular structure with a lateral body 32 with an open top end 31 and an open bottom end 33 further comprising a plurality of holes, openings, cutouts or slits 34 on the lateral body 32 as shown in one example in FIGS. 3-5 which allows disinfectant or IPA to move out from the sponge 20 to the recess or cavity 19 between the bellow 30 and the inner surface of the sidewall 18 of the rigid cap or housing 12. The lateral body 32 of the bellow 30 may comprise a plurality of ridges 36 and troughs 38 that may be compressed or deformed when the open female luer connector 112 engages the threads 17 on the rigid cap or housing 12 and thus applies a downward force on the bellow 30 and sponge 20. The bellow 30 may be comprised of thermoplastic polymer or elastomer, silicone, rubber, or other similar materials. The bellow 30 and sponge 20 may be bonded to the inner surface of the bottom 16 of the rigid cap 12. The plurality of ridges 36 and troughs 38 on the bellow 30 that may be sized or configured to fit within the threads 17 on the interior surface of the sidewall 18 of the rigid cap 12 to help retain the bellow 30 and thus the sponge 30 within the rigid cap 12. When the bellow 30 is compressed or deformed when the open female luer connector 112 engages the threads 17 on the rigid cap or housing 12, and thus applies a downward force on the bellow 30 and sponge 20, the plurality of ridges 36 and troughs 38 on the bellow 30 may align with and press against the threads 17 on the interior surface of the sidewall 18 of the rigid cap 12, thereby pushing disinfectant or IPA out of the sponge 20 and onto the threads 17 and into the recess 19 within the rigid cap 12 to further assist cleaning ad disinfecting the open female luer connector 112.

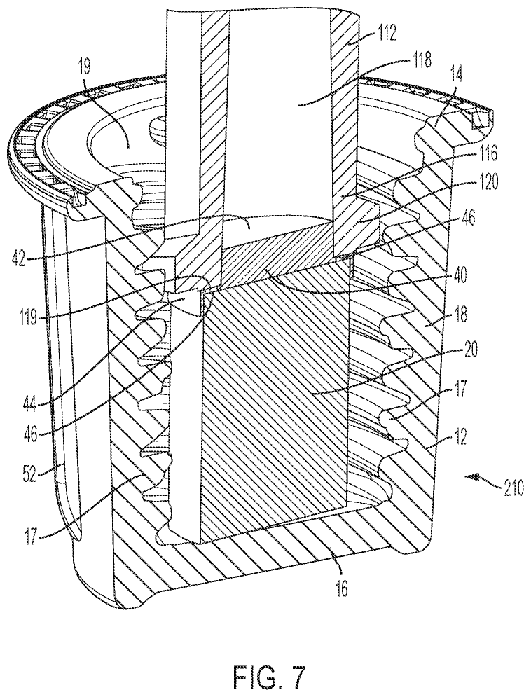

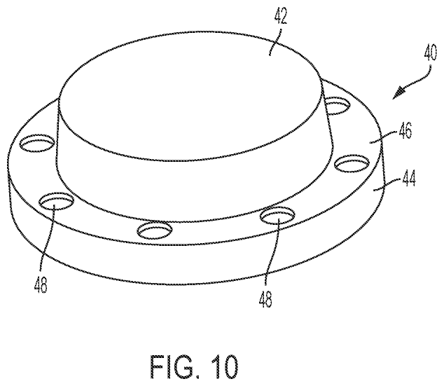

The cap 10 may further comprise a plug 40 that may be disc shaped as shown in FIGS. 2-5 and FIGS. 7-10. The plug 40 may act as a sealing element to seal or block the lumen 108 of the open female luer connector 112 to restrict or prevent ingress of the disinfectant or IPA into the lumen 108 and in turn into the fluid path of the open female luer connector 112 and the VAD. The plug may be comprised of a first round disc 42 of a smaller diameter formed or connected with a second round disc 44 of a larger diameter as shown in FIG. 10 configured such the first round disc 42 may be inserted into the lumen 118 of the open female luer connector 112 and the outer top surface 46 of the second round disc 44 that is of a greater diameter than the first round disc 42 may press against or seal the open distal end 116 and/or the distal face 119 of the open female luer connector 112 as shown for example in FIGS. 2 and 7. The plug 40 may further comprise in some examples a plurality of holes or openings 48 disposed on the outer top surface 46 of the second round disc 44 to allow for disinfectant or IPA to contact the distal face 119 of the open female luer connector 112. The plug 40 may be connected or bonded to or formed as part of the bellow 30 at the top open end 31 of the bellow 30 to at least partially close or seal the top open end 31 of the bellow 30. The bellow 30 formed with or connected to the plug 40 defines a recess or cavity in which the sponge 20 may be disposed.

The absorbent support or sponge 20, bellow 30, and other parts of the cap 10 of the present disclosure may be configured to cause the cleaning solution to contact multiple areas of the open female connector 112 ensuring that the multiple areas of the open female connector 110 remain sterile and free from microbes, contaminants, particles, and other debris. In particular, the cap 10 can be configured to ensure that cleaning solution contacts not only the outer surfaces of the distal end 116 of the open female luer connector 110 including the thread or luer lug 120, but also the distal face 119 of the open female luer connector 112. The IPA can be filled in the cap 10 and retained in the sponge 20 to act as a disinfectant.

In some examples, the rigid cap or housing 12 is a molded part formed by injection molding or other common molding processes. In some examples, the rigid cap or housing 12 can be formed from a rigid thermoplastic polymer material, such as polyester, polycarbonate, polypropylene, polyethylene, polyethylene terephthalate, or acrylonitrile butadiene styrene. Further, the rigid cap or housing 12 can be formed from a durable material, such as a material having a shore hardness D value of less than or equal to 95 (Shore D). Alternatively, the rigid cap or housing 12 can be formed from a more flexible material, such as a material having a shore hardness A value less than or equal to 130 (Shore A). Beneficially, a flexible housing 12 or housing 12 including flexible portions can be capable of stretching, bending, or otherwise deforming so that the cap 10 is capable of engaging open female luer connectors 112 of different sizes and shapes.

In some examples, the rigid cap or housing 12 further comprises protrusions, such as axially extending ribs or ridges 52, extending outward from an outer surface of the sidewall 18 of the housing 12. The axial ridges 52 can be provided to increase rigidity of the rigid cap 12 or housing 12 compared to if ridges 52 were not present. Also, the ridges 52 can make the rigid cap or housing 12 easier to grasp and manipulate improving usability of the cap 10 and, for example, making it less likely that the practitioner will drop or mishandle the cap 10 during use.

As shown most clearly in FIG. 2, the cap 10 further comprises the sponge 20 and bellow 30 disposed within the rigid cap or housing 12. As described in further detail herein, the sponge 20 and bellow 30 are flexible, deformable, or compressible structures configured to compress axially when the open female luer connector 112 is inserted into the rigid cap 12. In particular, the sponge 20 and below are configured to compress so that the distal end 116 of the open female connector 112 can be inserted farther into the rigid cap or housing 12 allowing thread or luer lug 130 of the connector 112 to come into contact with an engage the threads 17 on the inner surface of the sidewall 18 of the rigid cap 12. In some examples, the sponge 20 can be an annular, cylindrical, or tubular structure having an outer diameter that substantially matches (e.g., is the same as or within about 5% or less of) an inner diameter of the bellow 30 and less than the inner diameter of the rigid cap or housing 12.

The plug 40 may be inserted into the lumen 108 of the open female luer connector 112 and may press against or seal the open distal end 116 and/or the distal face 119 of the open female luer connector 112 as shown for example in FIGS. 2 and 7, which prevents contamination of lumen 108 of the open female luer connector 112.

In some examples, the sponge 20 comprises a sponge or a porous foam (e.g., an open cell foam) capable of absorbing the cleaning or disinfecting solution, such as a foam or sponge. The sponge 20 may also comprise a polyurethane a thermoplastic elastomer, such as polypropylene, polyethylene, or synthetic or natural rubber (e.g., isoprene). In other examples, the foam material can be a Plastazote® foam, which is an engineered polymer foam by Zotefoams PCL.

In some examples, the rigid cap 12 further comprises a plurality of threads 17 extending radially inward from the sidewall 18 of the rigid cap 12. The threads 17 can be configured to engage the thread or luer lug 130 on the distal end 116 of the open female luer connector 112 for securing the open female luer connector 112 to the rigid cap 12 and cap 10. Accordingly, the open female luer connector 112 can be securely retained within the rigid cap 12 by the threaded engagement preventing the cap 10 from being mistakenly removed from the open female luer connector 112 at unexpected or inappropriate times.

As previously described, the sponge 20 contains the cleaning solution for cleaning and/or disinfecting outer portions of the open female luer connector 112 engaged to the cap 10. As shown in FIG. 2, the sponge 20 can be disposed and held within a cylindrical interior space or cavity defined by the bellow 30, plug 40 and rigid cap 12. For example, sponge 20 can be held in place within the bellow 30 by a conventional adhesive or mechanical fastener. In other examples, the sponge 20 can be held in place in the bellow 30 by friction between an inner surface of the bellow 30 and an outer surface of the sponge 20. The bellow 30 can be disposed and held within the open-ended cylindrical recess or cavity defined by the sidewall 18 and bottom 16 of rigid cap 12. The bellow 30 may be held in place in the rigid cap 12 by friction between the outer surface of the bellow 30 and inner surface of the rigid cap. Specifically, the plurality of ridges 36 and troughs 38 on the bellow 30 that may be sized or configured to fit within the threads 17 on the interior surface of the sidewall 18 of the rigid cap 12 to help retain the bellow 30 and thus the sponge 30 within the rigid cap 12.

In some examples, the absorbent support or sponge 20 can be provided (i.e., presoaked) with the cleaning or disinfecting solution. For example, the cleaning or disinfecting solution can be an antimicrobial, anti-fungal, antibacterial, or antiviral solution that cleans and sterilizes outer surfaces of the open female luer connector 112. In some examples, the cleaning solution can be Isopropyl alcohol (IPA), such as about 70% IPA.

The absorbent support or sponge 20 and bellow 30 can be configured to axially compress as the distal end 116 of the female connector 112 is inserted into and moves through the interior of the cap 10 and housing 12. Axial compression of the absorbent support or sponge 20 and/or bellow 30 expels the cleaning solution from the absorbent support or sponge 20. The expelled cleaning solution moves through the recess 19 of the rigid cap or housing 12 towards the open top 14 of the rigid cap 12 contacting an outer surface 119 of the distal open end of the female luer connector 112 inserted into the rigid cap or housing 12. Once expelled from the absorbent support or sponge 20, the cleaning solution can also pass through openings, such as the slots 34, of the bellow 30. After passing through the slots 34, the cleaning solution comes into contact with other parts of the distal end 116 open female luer connector 112, such as the outer surface 119 and the threads or luer lugs 120. The cleaning solution can also pass through openings 42 in the plug 40, such that cleaning solution remains in proximity to the outer surface 119 and the surfaces of the threads or luer lugs 120 of the open female luer connector 112 for cleaning and disinfecting these surfaces.

In some examples, the cap 10 further comprises a plug 40 positioned in the recess 19 formed by the rigid cap or housing 12 over the absorbent support or sponge 20. The plug 40 can be configured to prevent fluids, such as cleaning or disinfecting solution absorbed by the absorbent support or sponge 20, from flowing into the lumen 118 of the open female luer connector 112.

The plug 40 can be sized and positioned so that cleaning solution expelled from the absorbent support or sponge 20 can move past the seal 44 towards the top 14 of the housing 12. As previously discussed, as the cleaning solution moves through the recess 19 formed by the rigid cap or housing 12, the cleaning solution contacts the outer surface 119 of the distal end 116 of the connector 112 for cleaning and disinfecting the outer surface. In order for the cleaning solution to move past the plug 40, an outer diameter 44 of the plug 40 can be larger another portion 42 of the plug 40 and the inner diameter of the lumen 118 of the connector 112 so that holes or openings 48 in the outer diameter 44 of the plug 40 allow the cleaning solution to contact the outer surface 119 of the connector 112 while also preventing the cleaning solution to ingress into the lumen 118 and flow path in the open female connector 112.

### Method for Attaching a Connector to the Cap

As previously described, the cap 10 of the present disclosure is a disinfecting cap 10 configured to be connected to various types and sizes of open female luer connectors 112. FIG. 2 shows the cap 10 connected to an open female luer connector 112 in one example.

In order to connect the cap 10 to an open female luer connector 112, the practitioner first removes any packaging from the cap 10. Once any packaging is removed, the practitioner moves the connector 112 towards the open top 14 of the rigid cap or housing 12. Specifically, the distal open end 114 of the open female luer connector 112 is inserted through the open top 14 of the housing 12 causing the distal open end 114 of the open female luer connector 112 to contact the plug 40 disposed in the housing, as shown for example in FIG. 2 and FIG. 6C. As shown in FIG. 2 and FIG. 6C, as the distal open end 114 of the open female luer connector 112 is inserted through the open top 14 of the housing 12, the threads or luer lugs 120 on the outer surface of the distal open end 114 of the of the open female luer connector 112 are brought into contact with the threads 17 of the rigid cap or housing 12. Also, contact between the distal open end 114 of the of the open female luer connector 112 and the plug 40 can block or seal off the lumen 118 extending through open female luer connector 112 preventing cleaning solution or other fluids from passing into the lumen 118 of the open female luer connector 112.

In order to move the open female luer connector 112 farther into the rigid cap or housing 12 of the cap 10, the practitioner next rotates the open female luer connector 112 relative to the cap 10 and housing 12. Rotation of the open female luer connector 112 relative to the housing 12 draws the open female luer connector 112 farther downward or into the cap 10, causing the distal open end 114 of the open female luer connector 112 to move through the interior of the rigid cap or housing 12. As the distal open end 114 of the open female luer connector 112 moves through the rigid cap or housing 12, it presses against the plug 40 which in turn axially compresses the absorbent support or sponge 20 thereby expelling the cleaning solution from the absorbent support or sponge into the interior of the rigid cap or housing 12, including in some examples through the openings or slots 34 in the bellow 30.

In some examples, the cleaning solution may move from the absorbent support or sponge 20, past the plug 40 and/or through the holes or openings 48 in the plug 40, and towards the open top 14 of the rigid cap or housing 12. As the cleaning solution is pushed through the rigid cap or housing 12, the cleaning solution can also pass through the holes or openings 42 in the plug 40, such that cleaning solution contacts the outer surface 119 and the surfaces of the threads or luer lugs 120 of the open female luer connector 112 for cleaning and disinfecting those surfaces.

The plug 40 can be positioned in the recess 19 formed by the rigid cap or housing 12 over the absorbent support or sponge 20 between the distal open end 114 of the open female luer connector 112 and the absorbent support or sponge 20. The plug 40 can be configured and is positioned to prevent fluids, such as cleaning or disinfecting solution absorbed by the absorbent support or sponge 20, from flowing into the lumen 118 of the open female luer connector 112. Specifically, the top portion 42 of the plug 40 advances into the lumen 118 through the distal open end 114 of the open female luer connector 112 thereby closing or sealing the lumen 118 by, for example, a friction fit between the interior sidewall of lumen 118 and the exterior surface of the top portion 42 of the plug 40.

Continued movement of the distal open end 116 of the open female luer connector 112 into the rigid cap or housing 12 causes the open female luer connector 112 to press against the cap 40 which in turn presses against and compresses the absorbent support 20 or sponge. The absorbent support or sponge 20 can also bulge radially outward pushing into the lateral body 32 of the bellow 30 which in turn may bulge radially outward until it contacts the inner surface of the sidewall 18 of the rigid cap or housing 12. The cleaning solution from the absorbent support or sponge 20 can also pass into the recess 19 defined by the interior of the rigid cap or housing and towards the open female luer connector 112 through the cutouts or slits 34 on the lateral body 32 of the bellow 30 for cleaning and disinfecting the distal end 118 of the open female luer connector 112.

In order to remove the cap 10 from the open female luer connector 112, the practitioner first rotates the open female luer connector 112 relative to the rigid cap or housing 12 causing the threads or luer lugs 120 of the open female luer connector 112 to back off of the threads 17 on the inner surface of the sidewall 18 of the rigid cap or housing 12. The rotation of the open female luer connector 112 relative to the rigid cap or housing 12 causes the open female luer connector 118 to begin to move axially upward out of the housing 12.

Once the threads or luer lugs 120 of the open female luer connector 112 are released from the threads 17 on the inner surface of the sidewall 18 of the rigid cap or housing 12, the practitioner can pull the open female luer connector 112 away from the rigid cap or housing 12, thereby releasing the open female luer connector 112 from the cap 10. For example, the practitioner can grasp the rigid cap or housing 12 of the cap 10 with one hand and the open female luer connector 112 with the other hand. The practitioner then pulls the open female luer connector 112 away from the cap 10 to remove the open female luer connector 112 from the cap 10. Once removed, the cap 10 can be discarded, as it is often a disposable single-use product.

Once the open female luer connector 112 is fully removed from the rigid cap or housing 12, the open female luer connector 112 can be connected to a VAD.

Disinfecting Cap with Disc Based Plug

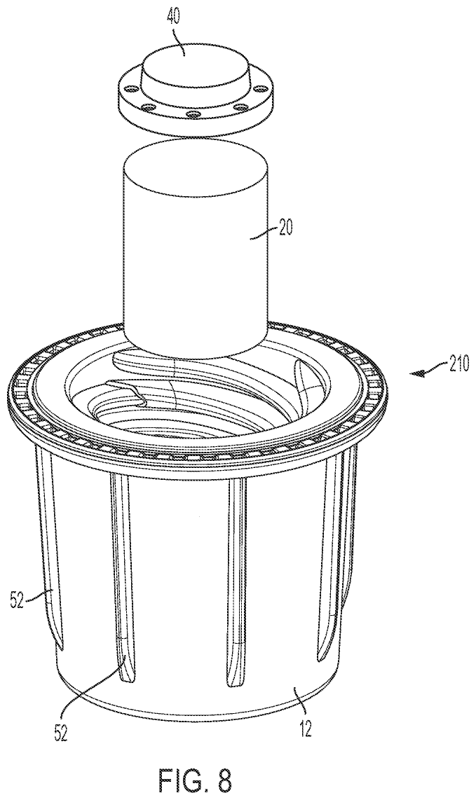

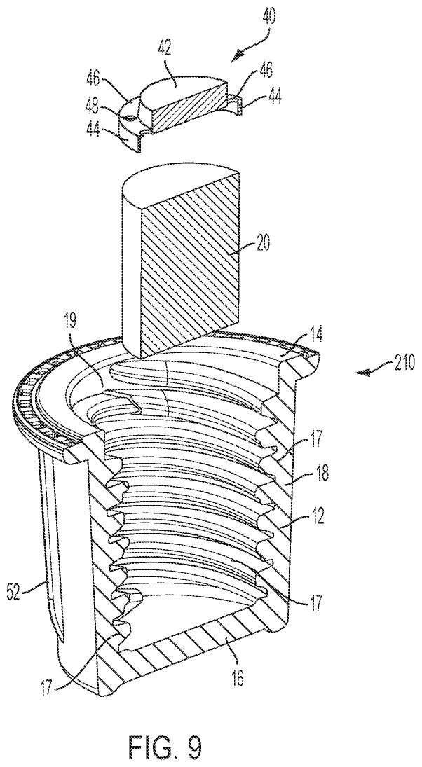

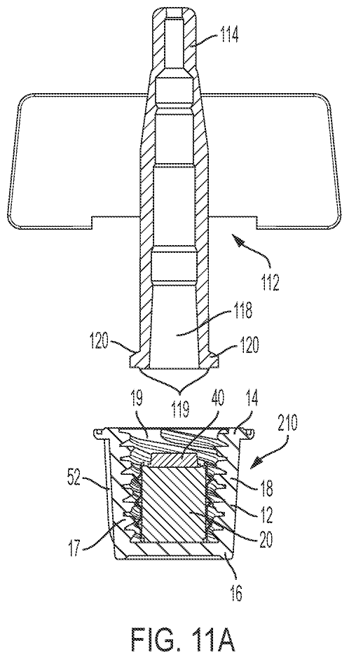

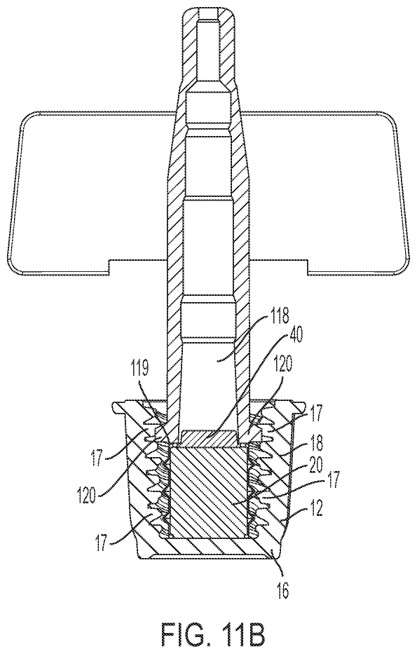

FIGS. 7-9 and FIGS. 11A-11B show another exemplary disinfecting cap 210 for an open female luer connector 110 including features of the present disclosure. Specifically, FIG. 7 shows a cross-sectional view of a cap 210 that comprises a rigid cap or housing 12, an absorbent support or sponge 20, and a plug 40. FIG. 8 shows an exploded view of the cap 210. FIG. 9 shows a cross-sectional view of the cap 210. FIG. 11A shows a cross-sectional view of the cap 210 and an open female luer connector 112, prior to inserting the connector 112 into the cap 210. FIG. 11B shows the open female luer connector 112 fully inserted into the cap 210.

The rigid cap or housing 12, the absorbent support or sponge 20, and the plug 40 from the cap 210 are configured the same as described in previous examples, such as shown in FIG. 2 for example, except that the cap 210 does not include a bellow 40. The cap 210 thus includes a housing 12 having an open first end or top 14, a second end or bottom 16, and a sidewall 18 extending between the top 14 and the bottom 16. The cap 210 also includes an absorbent support or sponge 20 disposed in the rigid cap or housing 12. Also, the cap 210 comprises the plug 40 configured to prevent the cleaning solution for cleaning and/or disinfecting portions of the open female connector 112 from be able to ingress or flow into the lumen 118 of the connector 112 as it is engaged to the cap 210. The plug 40 for the cap 210 may be a disc based plug 40 as shown in FIG. 10 as described previously.

In one example of cap 210, the plug 40 is axially aligned with and disposed over top of the absorbent support or sponge 20 and positioned within the recess 19 within the rigid cap our housing 12 as shown in FIGS. 11A-11B. The plug 40 may be configured to include a bottom disc portion 44 as shown in FIG. 10 which includes an inner diameter that is greater than the outer diameter of the absorbent support or sponge 20 such that the cap fits on top of and over the top of the absorbent support or sponge 20 as best viewed in FIGS. 11A-11B. The sponge 20 can be held in place within the housing 12 by a conventional adhesive or mechanical fastener and can be bonded or attached to the inner surface of the bottom 16 of the rigid cap 12. In other examples, the sponge 20 can be disposed and held within the open ended cylindrical recess or cavity 19 defined by the sidewall 18 and bottom 16 of rigid cap 12, including if the outer diameter of the absorbent support or sponge 20 is large enough by a friction fit with the sidewall 18 and/or threads 17 on the sidewall 17 of the rigid cap or housing 12.

As in previous examples, the rigid cap 12 of cap 210 further comprises a plurality of threads 17 extending radially inward from the sidewall 18 of the rigid cap 12. The threads 17 can be configured to engage the thread or luer lug 130 on the distal end 116 of the open female luer connector 112 for securing the open female luer connector 112 to the rigid cap 12 and cap 10. The absorbent support or sponge 20 can be configured to axially compress as the distal end 116 of the female connector 112 is inserted into and moves through the interior of the cap 210 and housing 12. Axial compression of the absorbent support or sponge 20 expels the cleaning solution from the absorbent support or sponge 20. The expelled cleaning solution moves through the recess 19 of the rigid cap or housing 12 towards the open top 14 of the rigid cap 12 contacting an outer surface 119 of the distal open end of the female luer connector 112 inserted into the rigid cap or housing 12. The cleaning solution can also pass through holes or openings 48 in the plug 40, such that cleaning solution remains in proximity to the outer surface 119 and the surfaces of the threads or luer lugs 120 of the open female luer connector 112 for cleaning and disinfecting these surfaces. The holes 48 on the top surface 46 of disc shaped plug 40 thus allow IPA to contact distal face 119 of open female luer connector 112 and disinfect this surface 119 as well.

In some examples, the cap 210 further comprises a plug 40 positioned in the recess 19 formed by the rigid cap or housing 12 over the absorbent support or sponge 20. The plug 40 can be configured to prevent fluids, such as cleaning or disinfecting solution absorbed by the absorbent support or sponge 20, from flowing into the lumen 118 of the open female luer connector 112.

The plug 40 can be sized and positioned in the housing 12 of cap 210 so that cleaning solution expelled from the absorbent support or sponge 20 can move past the plug 40 towards the top 14 of the housing 12. As previously discussed, as the cleaning solution moves through the recess 19 formed by the rigid cap or housing 12, the cleaning solution contacts the outer surface 119 of the distal end 116 of the connector 112 for cleaning and disinfecting the outer surface. In order for the cleaning solution to move past the plug 40, an outer diameter 44 of the plug 40 can be larger than another portion 42 of the plug 40 and the inner diameter of the lumen 118 of the connector 112 so that holes or openings 48 in the outer diameter 44 of the plug 40 allow the cleaning solution to contact the outer surface 119 of the connector 112 while also preventing the cleaning solution to ingress into the lumen 118 and flow path in the open female connector 112.

Disinfecting Cap with Coaxial Cylindrical Plug

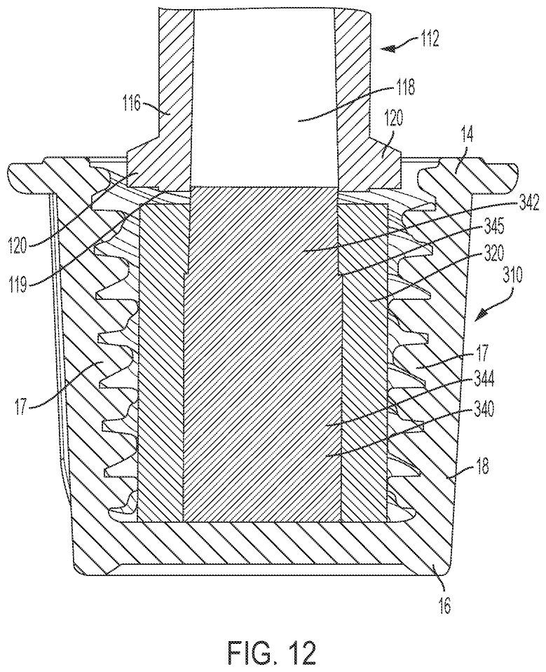

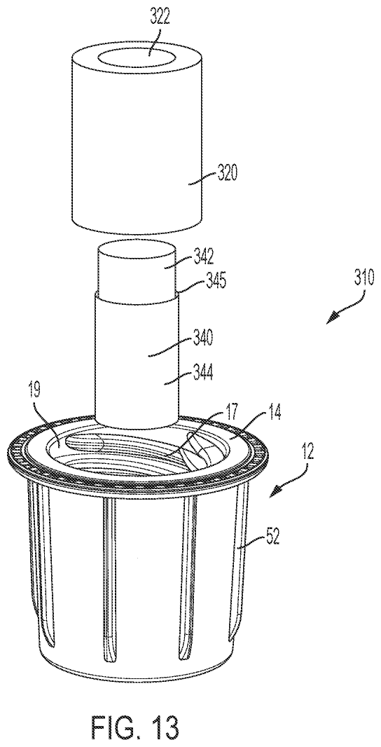

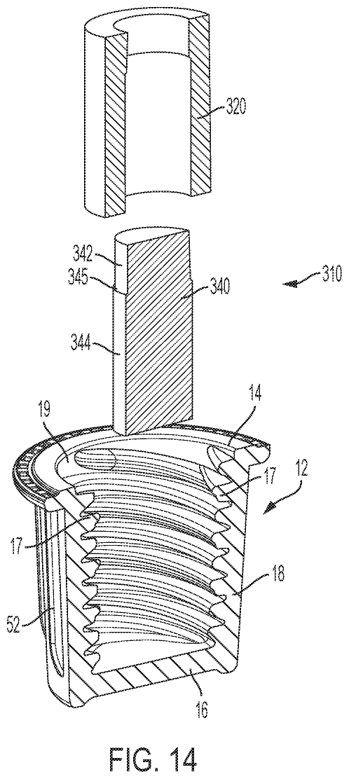

FIGS. 12-16 show another example of a cap 310 that includes features of the present disclosure wherein the cap 310 comprises a plug 340 that extends the full length of the sponge 320 and the sponge is in the form of an annular cylinder which fits around the plug 340. Specifically, FIG. 12 is a cross-sectional view of the cap 310 showing the open female luer connector 112 and the cap 310, after insertion of the connector 112 into the cap 310. FIG. 13 is an exploded perspective view of the cap 310 comprising a rigid cap or housing 12, a coaxial cylindrical plug 340, and a tubular absorbent support or sponge 320. The rigid cap or housing 12 is configured as set forth in previous examples as described in connection with the cap 12 shown in FIG. 2. The cap 310 thus comprises the housing 12 comprising the open first end or top 14, the closed second end or bottom 16, and the sidewall 18 extending between the top 14 and the bottom 16. The cap 310 also includes an absorbent support or sponge 320 disposed in the housing 12 that is tubular in shape and defines a cylindrical recess or opening 322 into which a coaxial cylindrical plug 340 may be inserted. The rigid cap or housing 12 is configured to engage the open female luer connector 112 for retaining the open female luer connector 112 within the housing 12. The absorbent support or sponge 320 disposed in the rigid cap or housing 12 and configured to surround the coaxial cylindrical plug 340 is configured to contain the cleaning solution for cleaning and/or disinfecting portions of the open female luer connector 112 engaged to the cap 310.

The absorbent support or sponge 320 can be constructed of the same materials and configured to operate in the same manner as previously described with respect to absorbent support or sponge 20. The absorbent support or sponge 320 can comprise disinfectant, such as IPA, for cleaning distal portions 116 of the open female luer male connector 112 when the connector 112 is engaged to the cap 310. The absorbent support or sponge 320 may be comprised of an open cell structure to retain IPA and release IPA when the absorbent support or sponge 320 is compressed by an incoming open female luer connector 112 when it is engaged to the cap 310, or more specifically, when the thread or luer lug 120 on the open female luer connector 112 engages the threads 17 on the rigid cap or housing 12. The absorbent support or sponge 320 can be configured to axially compress as the distal end 116 of the female connector 112 is inserted into and moves through the interior of the cap 310 and housing 12. Specifically, as shown in FIG. 16, the absorbent support or sponge 320 can be configured to directly contact the bottom surface 119 of the open distal end 116 and the threads or luer lugs 120 of the open female luer connector 112. Axial compression of the absorbent support or sponge 320 expels the cleaning solution from the absorbent support or sponge 320 such that it may contact and thus clean and disinfect the bottom surface 119 of the open distal end 116 and the threads or luer lugs 120 of the open female luer connector 112 as well as other portions of the outer surface of the distal end 116 of the open female luer connector 112.

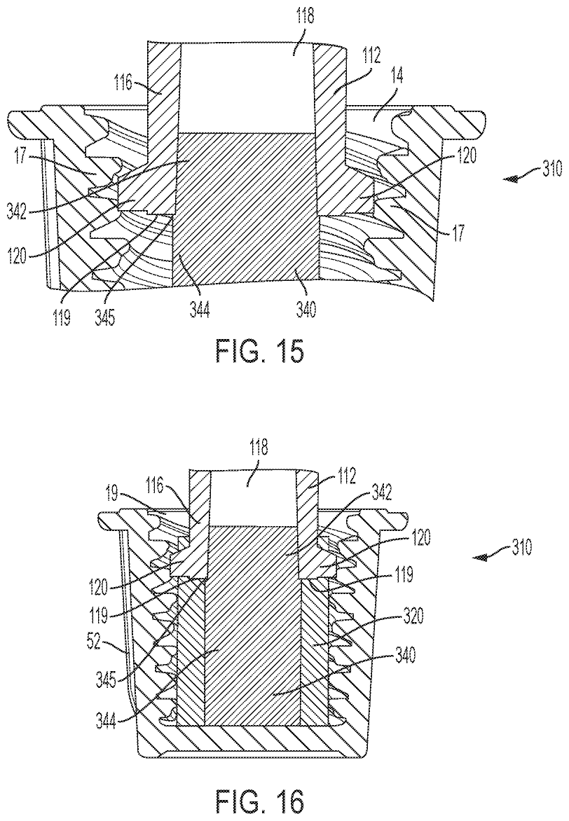

As shown in FIGS. 13-16, the plug 340 may comprise a first top solid cylindrical member 342 connected to or formed with a second bottom solid cylindrical member 344 wherein the first top solid cylindrical member 342 is of a smaller outer diameter than the second bottom solid cylindrical member 344. The top solid cylindrical member 342 may thus be sized in diameter to fit within the lumen 118 of the distal open end 116 of the open female luer connection 112 such that it can block or seal the lumen 118 from ingress of cleaning or disinfectant solution or IPA. The second bottom solid cylindrical member 34 of greater diameter may thus contact at its outer top edge 345, the bottom surface 119 of the open distal end 116 and the threads or luer lugs 120 of the open female luer connector 112 so that it can also block or seal the lumen 118 from ingress of cleaning or disinfectant solution or IPA as well as support the open female luer connector 112 in position within the rigid cap or housing 12 and/or prevent further axial movement of the open female luer connector 112 into the recess 19 formed within the rigid cap or housing 12 as best shown in FIGS. 15-16.

As shown in FIGS. 12, 15, and 16, as the distal end 116 of the open female luer connector 112 is inserted into the open end 14 of the rigid cap or housing 12, the first top solid cylindrical member 342 of the plug 340 can be configured to extend further beyond the top of the absorbent support or sponge 320 such that the first top solid cylindrical member 342 of the plug 340 is inserted into the lumen 118 of the open female luer connector 112, thereby blocking or sealing the lumen 118, before the distal end 116 of the open female luer connector 112 contacts and thus compresses the absorbent support or sponge 320. The cleaning or disinfectant solution pushed or squeezed out of the absorbent support or sponge 320 by axial compression of the absorbent support or sponge 320 is thus prevented from ingress into the lumen 118.

Disinfecting Cap with Hemispherical Plug

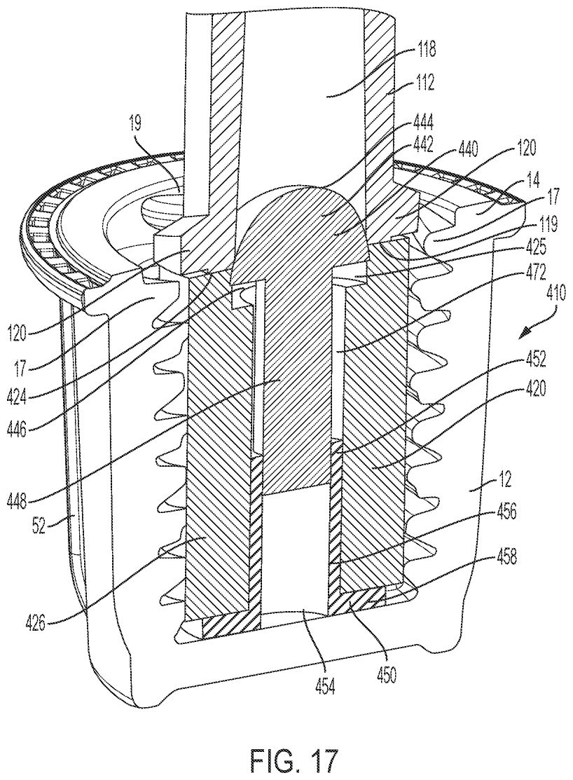

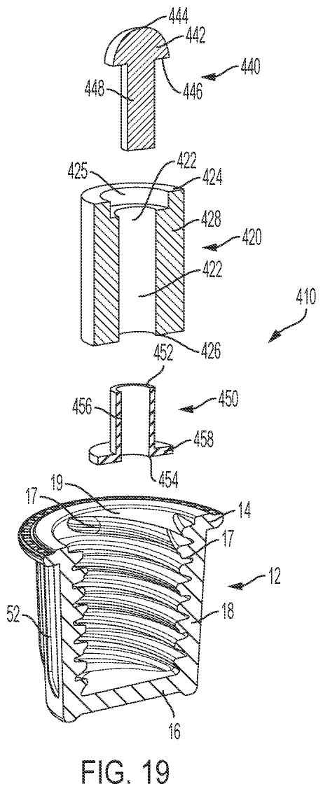

The cap 410 shown in FIGS. 17-20B differs from previous examples in that the cap 410 comprises a hemispherical plug 440, a plug guide 450, and a coaxial absorbent support or sponge 420 in addition to a rigid cap or housing 12 as configured and shown in previous examples. The rigid cap or housing 12 can be configured as set forth in previous examples as described in connection with the cap 12 shown for example in FIG. 2. The coaxial absorbent support or sponge 420 can be constructed of the same materials and configured to operate in the same manner as previously described with respect to absorbent support or sponge 20. The plug 440 in this example is pushed down into the plug guide 450 by connecting the female luer connector 112 to the cap 410, but the plug 440 keeps pressing against the distal face of the female luer connector 112 and its lumen 118.

As shown in FIGS. 17-2B, the plug guide 450 comprises an open first end or top 452, an open second end or bottom 454, and a sidewall 456 extending between the top 452 and the bottom 454. The plug guide can further comprise an annular disc shaped base 458 extending from the outer surface of the sidewall 456 of the plug guide 450 adjacent the bottom 454 of the plug guide 450. The plug guide 450 can be part of, affixed or connected to the rigid cap or housing 12 and can comprise a single molded plastic piece in some examples.

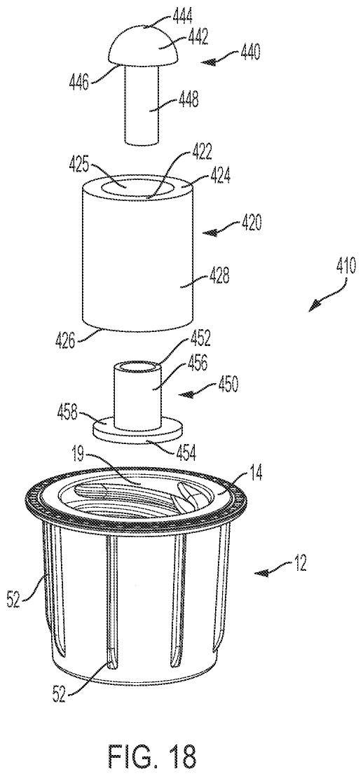

As shown in FIGS. 18-20B, the hemispherical plug 440 comprises a top hemispherical member 442 that comprises one-half of a sphere with a rounded, spherical top portion 444 and a flat bottom portion 446, and a bottom cylindrical member 448 which is connected, formed or molded to the flat bottom portion 446 of the top hemispherical member 442. The bottom cylindrical member 448 is configured to be inserted into the first open end or top 452 of the plug guide 450. The bottom cylindrical member 448 may be affixed to or connected via a friction fit with the first open end or top 452 of the plug guide 450. The top hemispherical member 442 and spherical top portion 444 of the hemispherical plug 440 may be configured and sized in diameter to fit within or conform to the lumen 118 of the distal open end 116 of the open female luer connection 112 such that it can block or seal the lumen 118 from ingress of cleaning or disinfectant solution or IPA. The bottom cylindrical member 448 is configured and sized to fit within the cylindrical opening 422 in the center of the absorbent support or sponge 420.

As shown in FIGS. 18-20B, the absorbent support or sponge 420 may comprise a top open end 424, a bottom open end 426, and sidewall 428 that forms or defines a tubular shaped structure with a cylindrical opening 422 running through the middle of absorbent support or sponge 420. The open top end 424 may have a larger diameter opening 425 than the cylindrical opening 422 to contact or support the flat bottom portion 446 of the hemispherical plug 440. The absorbent support or sponge 320 can be constructed of the same materials and configured to operate in the same manner as previously described with respect to absorbent support or sponge 20.

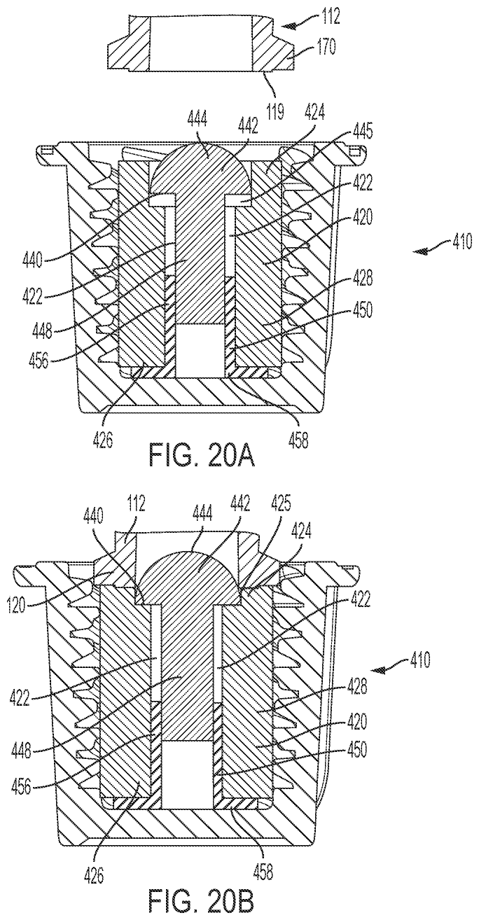

The absorbent support or sponge 420 can comprise disinfectant, such as IPA, for cleaning distal portions 116 of the open female luer male connector 112 when the connector 112 is engaged to the cap 410. The absorbent support or sponge 420 may be comprised of an open cell structure to retain IPA and release IPA when the absorbent support or sponge 420 is compressed by an incoming open female luer connector 112 when it is engaged to the cap 410, or more specifically, when the thread or luer lug 120 on the open female luer connector 112 engages the threads 17 on the rigid cap or housing 12. The absorbent support or sponge 420 can be configured to axially compress as the distal end 116 of the female connector 112 is inserted into and moves through the interior of the cap 310 and housing 12. Specifically, as shown in FIG. 17 and FIG. 20B, the absorbent support or sponge 420 can be configured to directly contact the bottom surface 119 of the open distal end 116 and the threads or luer lugs 120 of the open female luer connector 112. Axial compression of the absorbent support or sponge 420 expels the cleaning solution from the absorbent support or sponge 320 such that it may contact and thus clean and disinfect the bottom surface 119 of the open distal end 116 and the threads or luer lugs 120 of the open female luer connector 112 as well as other portions of the outer surface of the distal end 116 of the open female luer connector 112.

As shown in FIGS. 17 and FIGS. 20A-20B, as the distal end 116 of the open female luer connector 112 is inserted into the open end 14 of the rigid cap or housing 12, the top hemispherical member 442 with a rounded, spherical top portion 444 of the hemispherical plug 440 can be configured to extend further beyond the top 424 of the absorbent support or sponge 420 such that the top hemispherical member 442 with a rounded, spherical top portion 444 of the hemispherical plug 440 is inserted into the lumen 118 of the open female luer connector 112, thereby blocking or sealing the lumen 118, before the distal end 116 of the open female luer connector 112 contacts and thus compresses the absorbent support or sponge 420. The cleaning or disinfectant solution pushed or squeezed out of the absorbent support or sponge 420 by axial compression of the absorbent support or sponge 420 is thus prevented from ingress into the lumen 118.

### Plugs for Disinfecting Cap

As previously described, the plugs 40, 340, 440 for the disinfecting caps 10, 210, 310, and 410 disclosed herein are all configured to block or seal the lumen 118 of the open female luer connector 112 with an interference fit. The plugs 40, 340, and 440 are configured and positioned within the disinfecting caps 10, 210, 310, and 410 to reduce the chances of disinfectant or IPA ingress into the lumen 118 of the open female luer connector 112.

While examples of the disinfecting caps 10, 210, 310, 410 and methods of use of the caps as set forth in the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A disinfectant cap configured to engage an open female luer connector comprising a lumen, the cap comprising:
a housing comprising an open first end, a second end, and at least one sidewall extending between the first end and the second end defining a recess;
a plug disposed in the recess of the housing configured to engage the open female luer connector for sealing the lumen of the open female connector; and
an absorbent support disposed in the recess of the housing configured to contain a cleaning solution for cleaning and/or disinfecting portions of the open female luer connector engaged to the cap.

2. The cap of claim 1, wherein the absorbent support comprises a sponge.

3. The cap of claim 1, wherein the cleaning solution comprises at least one of Isopropyl Alcohol (IPA), chlorhexidine, and povidone-iodine.

4. The cap of claim 1, wherein, when the open female luer connector is engaged to the cap, cleaning solution is exposed to portions of the open female luer connector for cleaning and/or disinfecting the open female luer connector.

5. The cap of claim 1, wherein the open female luer connector further comprises an outer surface and a thread extending from the outer surface and the sidewall of the housing further comprises an inner surface that comprises threads to engage the thread on the outer surface open female luer connector.

6. The cap of claim 1, further comprising a bellow comprising a first open end, a second open end, and a sidewall defining a cylindrical opening that is configured to enclose the absorbent support wherein the first open end of the bellow is configured to connect to the plug to further enclose the absorbent support.

7. The cap of claim 6, wherein the bellow further comprises a sidewall comprising ridges and troughs configured to be compressed and a plurality of openings in the sidewall configured to allow cleaning solution to flow from the absorbent support into the recess formed by the housing to clean and disinfect portions of the open female luer connector when the absorbent support is compressed by the insertion of the open female luer connector into the cap.

8. The cap of claim 7, wherein the cleaning solution comprises at least one of Isopropyl Alcohol (IPA), chlorhexidine, or povidone-iodine and the absorbent support comprises a sponge.

9. The cap of claim 1, wherein the plug comprises a first disc configured to fit within and seal the lumen of the open female luer connector.

10. The cap of claim 9, wherein the plug further comprises a second disc having a larger diameter than the first disc and connected to the first disc wherein the second disc comprises a plurality of holes for permitting the cleaning solution to move past the plug towards the open first end of the housing.

11. The cap of claim 1, wherein the plug comprises a hemispherical member configured to fit within and seal the lumen of the open female luer connector.

12. The cap of claim 11, wherein the plug further comprises a cylindrical member with a first end affixed to the hemispherical member and a second end slidably engaged to a plug guide comprising a top open end for receiving the second end of the cylindrical member of the plug and a bottom second end connected to the second end of the housing.

13. The cap of claim 12, wherein the absorbent support comprises an open top end, an open bottom end, and sidewall extending between the top and bottom ends defining an inner cylindrical opening configured to receive and surround portions of the plug.

14. The cap of claim 1, wherein the plug comprises a cylindrical member comprising a first end configured to fit within and seal the lumen of the open female luer connector and a second end having a larger diameter than the first end and the lumen configured to further engage the open female luer connector.

15. The cap of claim 14, wherein the absorbent support comprises an open top end, an open bottom end, and sidewall extending between the top and bottom ends defining an inner cylindrical opening configured to receive and surround portions of the second end of the plug.

16. The cap of claim 1, wherein insertion of the open female luer connector into the housing causes the absorbent support to axially compress farther into the housing, and
wherein the axial compression of absorbent support expels the cleaning solution from the absorbent support causing the cleaning solution to move through an interior of the housing contacting an outer surface of the open female luer connector inserted into the housing.

17. A method for attaching the cap of claim 1 to the open female luer connector, the method comprising:
inserting a distal open end of the open female connector through the open first end of the housing causing the proximal end of the open female connector to contact the plug disposed in the housing and the absorbent support; and
rotating the open female luer connector relative to the housing causing the proximal end of the open female luer connector to advance through the housing axially compressing the absorbent support.

18. A cap configured to engage an open female connector comprising a lumen, the cap comprising:
a housing comprising an open first end, a second end, and at least one sidewall extending between the first end and the second defining a recess;
a bellow comprising an open first end, an open second end, and at least one sidewall extending between the first end and the second end defining a cavity disposed in the recess defined by the housing;
an absorbent support disposed in the cavity defined by the bellow and contained by the sidewall of the bellow configured to contain a cleaning solution for cleaning and disinfecting portions of the female connector engaged to the cap;
a plug connected to the open first end of the bellow configured to further contain the absorbent support and prevent the cleaning solution from entering the lumen of the female connector.

19. The cap of claim 18 wherein the absorbent support comprises a sponge and the cleaning solution is a disinfecting liquid such as but not limited to Isopropyl Alcohol (IPA), chlorhexidine, and povidone-iodine.

20. A cap configured to engage a female connector comprising a lumen, the cap comprising:
a rigid housing comprising an open first end, a second end, and a sidewall extending between the first end and the second defining a recess;
a sponge disposed in the recess defined by the housing configured to contain cleaning solution comprising at least one of Isopropyl Alcohol (IPA), chlorhexidine, or povidone-iodine for cleaning and disinfecting portions of the female connector engaged to the cap;
a plug configured to be inserted into the lumen of the female connector to prevent IPA from entering the lumen of the female connector.
